(19) United States
(12) Patent Application Publication
(10) Pub. No.: US 2020/0088618 A1
(43) Pub. Date: Mar. 19, 2020

(54) METHODS AND SYSTEMS FOR MEDICAL GAS ASSET INSPECTION AND COMPLIANCE

(71) Applicants: Daniel Timothy Voight, Lakeland, FL (US); Laura Isabel Lopes, Lakeland, FL (US)

(72) Inventors: Daniel Timothy Voight, Lakeland, FL (US); Laura Isabel Lopes, Lakeland, FL (US)

(21) Appl. No.: 16/135,649

(22) Filed: Sep. 19, 2018

(51) Int. Cl. G01N 7/00; G06F 16/955

(52) U.S. Cl. CPC G01N 7/00; G01N 33/0062; G06F 16/9558

(57) ABSTRACT

Some implementations can include a computer-implemented method and/or system for medical gas inspection and electronic inspection report generation.

### FIELD

Some implementations relate to the field of conducting inspections. More specifically, some implementations relate to the implementation of a facilitating web-based computer system and processes for creating and utilizing medical gas inspection forms, and in automatically incorporating sensor measurements and other information along with the inspection forms presented.

### BACKGROUND

Some facilities, such as medical facilities, may require periodic inspections of various systems such as medical gas systems. In some instances, these inspections are intended to detect an asset condition and performance. A typical site may include thousands of various components which need to be independently evaluated to make sure they still have the desired properties. For example, where the property is medical gas provision within specified parameters, a particular asset, e.g., gas outlet or vacuum outlet will be inspected, measured and detailed notes and measurement taken regarding properties relating to the type, location, condition and one or more operational parameters of the particular asset.

It is known for some inspectors to use portable computing devices to assist with the inspection process. In doing so, these inspectors may take either paper or electronic Adobe copies of the site schematics (e.g., plant) so that they can find locations of the assets to be inspected. Once located, the inspectors visually inspect the various components/assets and document the condition of different assets, e.g., medical gas equipment, etc. This is sometimes done by recording the visual inspection results into a report, which can be saved after completion into the computing device. The inspector may also take photographs to document the condition of the assets.

Once the onsite work is completed, the inspector typically returns to the office, and completes a comprehensive report using the information obtained on the platform or other site. Also, the inspector will somehow associate the photos taken with whatever information is related to the same assets which were photographed. This takes considerable time, but in the end, the report is completed, and kept for future reference.

If, in the future, reference need be made of the report, the interested party uses the hard copy site schematics for the purpose of locating the assets, and then must engage in a matching up of the asset components evaluated in the old report against those shown in the hard copy site schematic. This makes the process of comparing changes in degradation from one inspection to the next a time consuming and sometimes error prone ordeal.

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

### SUMMARY

Some implementations can include a tool for conducting medical gas inspections. The tool can include an inspection-item presenting process operating on a computing device, the inspection-item presentation process causing the display of an inspection form corresponding to a selected medical gas asset from among a plurality of medical gas assets being inspected as a facility, the selection of a given asset bringing up a list of selectable inspection items related to the asset, each inspection item being associated with an identifier, wherein each asset is associated with a zone of the medical gas system within the facility.

The tool can also include an information-gathering process also initiated upon the selection of a particular inspection item having a particular identifier, the information-gathering process opening up an inspection form for the particular inspection item selected on the computing device, the form posing standardized questions relating to at least one condition relating to one or more medical gas performance measurements of the given asset, the standardized questions made to be unalterable by a user of the computing device.

The tool can also include an operational pressure test rig interfacing process enabling the computing device to receive medical gas asset performance readings into the inspection form. The tool can further include an operations and maintenance manual linking process to link assets in an electronic inspection report with a corresponding operation and maintenance manual such that the link in the electronic inspection report is a hyperlink that permits a user to select the link and automatically access the corresponding operation and maintenance manual section for a corresponding asset.

### BRIEF DESCRIPTION OF THE DRAWINGS

FIG. 1 is a block diagram of an example medical gas inspection system and a network environment in accordance with some implementations.

FIG. 2 is a flowchart for medical gas inspections in accordance with some implementations.

FIG. 3 is flowchart for medical gas inspection report generation in accordance with some implementations.

FIG. 4 is diagram of an operational pressure test rig with gas specific adapter in accordance with some implementations.

FIG. 5 is a diagram of an example purity meter in accordance with some implementations.

FIG. 6 is a block diagram of an example computing device in accordance with some implementations.

FIG. 7 is a diagram of an example electronic inspection report in accordance with some implementations.

### DETAILED DESCRIPTION

The systems and methods provided herein may overcome one or more deficiencies of some conventional medical gas inspection and reporting techniques.

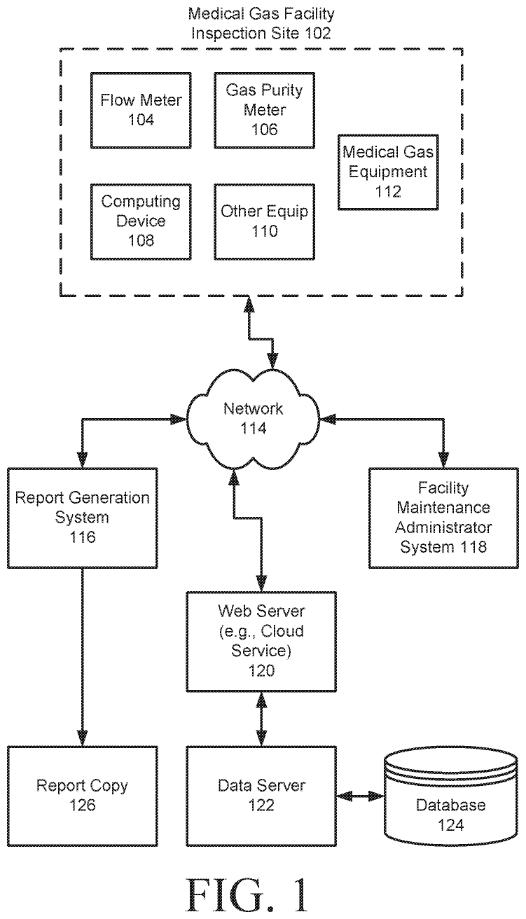

FIG. 1 is a block diagram of an example medical gas inspection system and a network environment in accordance with some implementations. A medical gas facility inspection site 102 (e.g., a hospital, clinic or other health care facility where medical gas is utilized can include medical gas equipment 112. The medical gas equipment can include equipment for the storage and delivery of medical gas such as medical air, nitrous oxide, oxygen, carbon dioxide, nitrogen, etc. As used herein, medical gas equipment also includes vacuum equipment. Medical gas equipment can include medical gas/vacuum outlets, manifolds, storage tanks, generation/concentration equipment, etc.

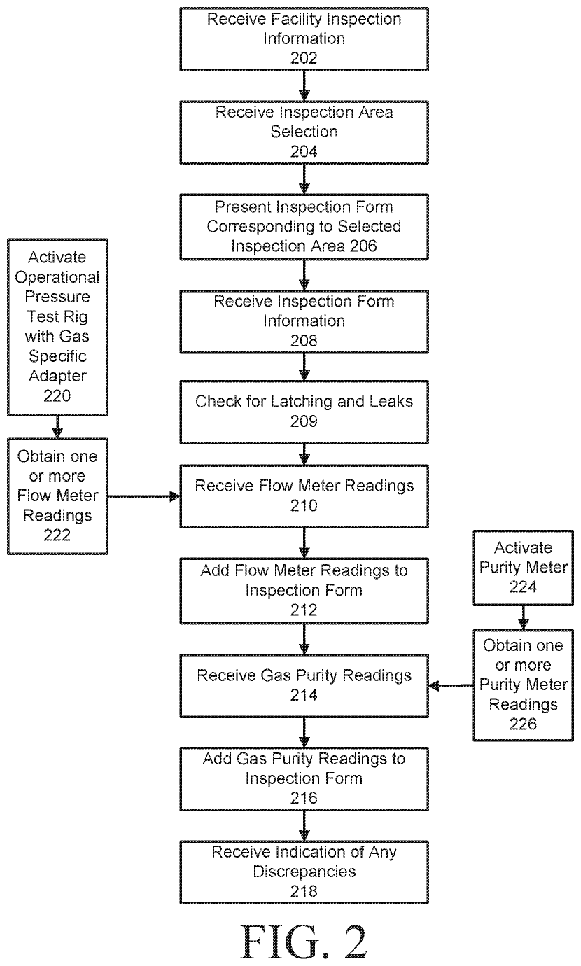

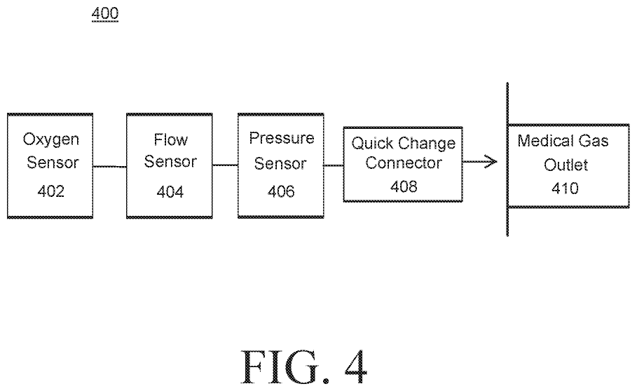

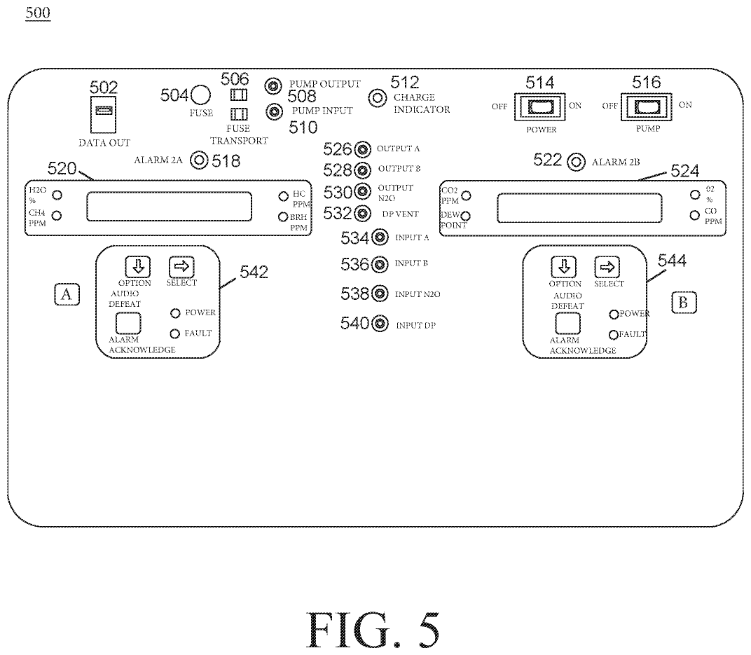

During an inspection of the medical gas facility inspection site 102, an inspector may use an operational pressure test rig 104 (e.g., similar to that shown in FIG. 4 and described below), a gas purity measuring device (e.g., similar to that shown in FIG. 5 and described below), a computing device (e.g., laptop or tablet computer), and/or other equipment 110. At the inspection site 102, the inspection system can perform an inspection process such as that shown in FIG. 2 and described below.

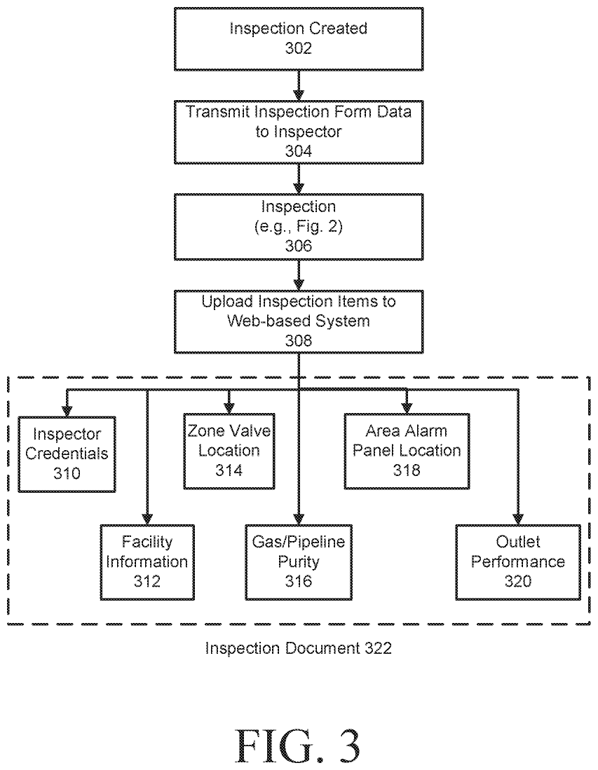

One or more of the inspection devices (e.g., 104-110) can communicate with other systems via a network 114 (e.g., the Internet) that can include a wired or wireless network or a combination of the above. The other systems can include a report generation system 116 (e.g., a system used to perform the process shown in FIG. 3, for example), a facility maintenance administrator system 118 (e.g., associated with an inspection site such as 102), or a web server 120 (e.g., cloud computing service). The web server 120 can be connected to a database server 122 and a database 124 (e.g., a cloud storage system) to store inspection results and/or reports, etc.

FIG. 2 is a flowchart for a medical gas inspection method in accordance with some implementations. Processing begins at 202, where facility inspection information is received. The facility inspection information can include details of an inspection site such as building locations, medical gas asset lists, locations of medical gas assets to be inspected, etc. Processing continues to 204.

At 204, the system can receive a selection of an inspection area. For example, a selection of an area (e.g., a zone valve area within an inspection site) can be made via a graphical user interface and the selection made via the graphical user interface can be received by the inspection system. Processing continues to 206.

At 206, an inspection form (e.g., similar to that shown in Appendix A) corresponding to the selected area is displayed on a display of a computing device. Processing continues to 208.

At 208, inspection form information is received. The inspection form information can include inspection location (e.g., received via GPS associated with inspection computing device) and computer readable information corresponding to an asset being inspected (e.g., barcode, RFID tag, etc., associated with a medical gas asset being inspected). Processing continues to 209. Separately from the processing, at 220, an operational pressure test rig is connected to a medical gas outlet and the test rig is activated.

At 209, an operational pressure test rig can be connected to a medical gas outlet being inspected to check for latching and leaks. Any problems with latching or leaks are noted in the inspection report being displayed by the inspection computing device. Processing continues to 210.

Separately from the processing, at 222, one or more sensor readings are obtained.

At 210, sensor readings are received from the operational pressure test rig. The sensor readings can include pressure (or vacuum), flow rate, or oxygen concentration, among other things. Processing continues to 212.

At 212, the operational pressure test readings are added to the inspection form. Processing continues to 214.

Separately from the processing, at 224, a purity meter is activated, and, at 226, one or more purity meter readings are obtained.

At 214, one or more gas purity sensor readings are obtained (e.g., from medical gas purity measurement equipment such as that shown in FIG. 5 and described below). Processing continues to 216.

At 216, the medical gas purity readings are added to the inspection form. Processing continues to 218.

At 218, any indication of discrepancies are obtained and optionally displayed to a user.

FIG. 3 is flowchart for medical gas inspection report generation in accordance with some implementations. Processing begins at 302, where an inspection is created (e.g., using a report generation system such as 116). The inspection can include details about the inspection site and inspection forms corresponding to one or more medical gas assets at the site. Processing continues to 304.

At 304, inspection form data is electronically transmitted to an inspector system (e.g., 108). Processing continues to 306.

At 306, an inspection process is performed (e.g., similar to that shown in FIG. 2 and described above). Processing continues to 308.

At 308, the inspection information (e.g., one or more completed inspection forms) is uploaded to a web-based inspection system (e.g., 116). Processing continues to 310.

The web-based inspection system can generate an inspection document 322 (e.g., similar to the form shown in Appendix A) that can include inspector credentials 310 (e.g., inspector name and license or certification information), facility information 312 (e.g., facility location, inspection site information, etc.), zone valve location 314, gas/pipeline purity 316, area alarm panel location 318, and/or medical gas outlet performance 320.

FIG. 4 is diagram of an operational pressure test rig 400 with gas specific adapter 408 in accordance with some implementations. The test rig 400 can include one or more of an oxygen sensor 402 to measure concentration of oxygen, a flow sensor 404 to measure the rate of flow, a pressure sensor 406 to measure pressure (or vacuum), and a gas specific adapter 408 that can serve as a kind of quick change connector for connecting one of a variety of gas specific connectors to the operational pressure test rig. The gas specific adapter/quick change connector 408 can have a gas specific connector attached to it to couple with a medical gas outlet 410.

FIG. 5 is a diagram of an example purity meter 500 in accordance with some implementations. The meter 500 can include a data port 502 for sending gas purity measurement data to an inspection system. The meter 500 can also include a fuse 504, fuse transports 506, a pump output connector 508 and a pump input connector 510. The meter 500 can also include a charge indicator 512, a power switch 514 and a pump activation switch 516.

The meter 500 can also include a first alarm 518 and a first display 522, a second alarm 522 and a second display 524. The meter 500 can include connections such as an output A 526, an output B 528, a N2O output 530, a DP vent connection 532, an input A 534, an input B 536, an N2O input 538, and/or an input DP 540. The meter 500 can include a first control panel 542 and a second control panel 544, where each control panel includes controls for selecting options, acknowledging an alarm, power and fault indicators.

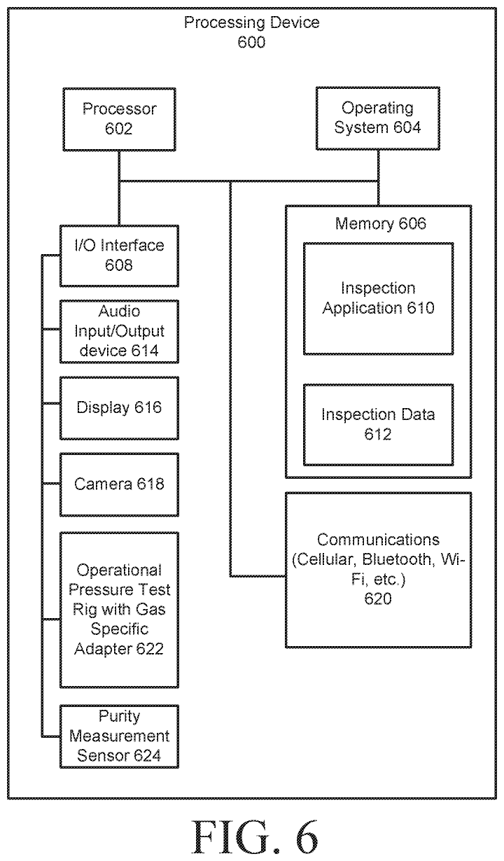

FIG. 6 is a block diagram of an example processing device 600 which may be used to implement one or more features described herein. In one example, device 600 may be used to implement a computer device, e.g., a server device (e.g., server device 108 of FIG. 1), and perform appropriate method implementations described herein (e.g., one or more of 202-226). Device 600 can be any suitable computer system, server, or other electronic or hardware device. For example, the device 600 can be a mainframe computer, desktop computer, workstation, portable computer, laptop computer, or electronic device (portable device, mobile device, cell phone, smart phone, tablet computer, television, TV set top box, personal digital assistant (PDA), media player, game device, wearable device, etc.). In some implementations, device 600 includes a processor 602, an operating system 604, a memory 606, and input/output (I/O) interface 608.

Processor 602 can be one or more processors and/or processing circuits to execute program code and control basic operations of the device 600. A “processor” includes any suitable hardware and/or software system, mechanism or component that processes data, signals or other information. A processor may include a system with a general-purpose central processing unit (CPU), multiple processing units, dedicated circuitry for achieving functionality, or other systems. Processing need not be limited to a particular geographic location, or have temporal limitations. For example, a processor may perform its functions in “real-time,” “offline,” in a “batch mode,” etc. Portions of processing may be performed at different times and at different locations, by different (or the same) processing systems. A computer may be any processor in communication with a memory.

Memory 606 is typically provided in device 600 for access by the processor 602, and may be any suitable processor-readable storage medium, e.g., random access memory (RAM), read-only memory (ROM), Electrical Erasable Read-only Memory (EEPROM), Flash memory, etc., suitable for storing instructions for execution by the processor, and located separate from processor 602 and/or integrated therewith. Memory 606 can store software operating on the server device 600 by the processor 602, including an operating system 604, one or more applications 610, and data 612. In some implementations, applications 610 can include instructions that enable processor 602 to perform the functions described herein, e.g., some or all of the methods of FIG. 2 or 3.

For example, applications 610 can include an inspection application. Other applications or engines 614 can also or alternatively be included in applications 610, e.g., email applications, SMS and other phone communication applications, web browser applications, media display applications, communication applications, web hosting engine or application, social networking engine or application, etc. Any of software in memory 604 can alternatively be stored on any other suitable storage location or computer-readable medium. In addition, memory 804 (and/or other connected storage device(s)) can store images, video, and other instructions and data used in the features described herein. Memory 604 and any other type of storage (magnetic disk, optical disk, magnetic tape, or other tangible media) can be considered “storage” or “storage devices.”

I/O interface 608 can provide functions to enable interfacing the server device 600 with other systems and devices. For example, network communication devices, storage devices (e.g., memory and/or database), and input/output devices can communicate via interface 608. In some implementations, the I/O interface 608 can connect to interface devices including input devices (keyboard, pointing device, touchscreen, microphone, camera, scanner, etc.) and/or output devices (display device, speaker devices, printer, motor, etc.). Audio input/output device 614 (e.g., microphone and speaker), display device 616 and camera device 618 are examples of input/output devices that may be used to capture input (microphone and/or camera) and to provide output (display and speaker). Display device 616 can be connected to device 600 via local connections (e.g., display bus) and/or via networked connections and can be any suitable display device, some examples of which are described below.

For ease of illustration, FIG. 6 shows one block for each of processor 602, memory 606, I/O interface 608, and software block 610. These blocks may represent one or more processors or processing circuitries, operating systems, memories, I/O interfaces, applications, and/or software modules. In other implementations, device 600 may not have all of the components shown and/or may have other elements including other types of elements instead of, or in addition to, those shown herein. While computing system 108 is described as performing operations as described in some implementations herein, any suitable component or combination of components of system 102 or similar system, or any suitable processor or processors associated with such a system, may perform the operations described.

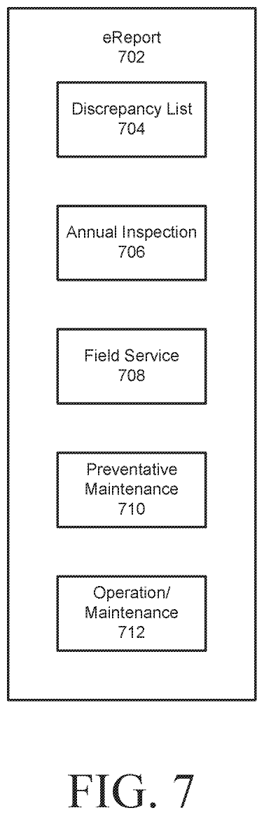

FIG. 7 is a diagram of an example electronic inspection report 702 in accordance with some implementations. The electronic report 702 can include a discrepancy list 704, which can include a list of any discrepancies noted during an inspection with an optional hyperlink to a work order for repair of the discrepancy.

The electronic report 702 can also include an annual inspection 706. The annual inspection 706 can include an inspection document (e.g., 322) such as a zone valve/area alarm/outlet performance/gas concentration and purity inspection document for each outlet in a given zone. The document can include zone valve location information and testing data, gas/pipeline purity test data (e.g., data provided by purity tester such as 500), area alarm panel location and testing data, outlet location (e.g., room number) and flow, pressure, and outlet type data (e.g., gathered from operational pressure test rig).

The electronic report 702 can also include field service information 708 providing information about field service procedures. The electronic report 702 can also include preventative maintenance information 710.

The electronic report 702 can also include operation/maintenance information (e.g., an electronic version of operation and maintenance manual. The operation and maintenance manual can be hyperlinked to from inspection information. For example, an outlet type in the inspection form can be a hyperlink to the operation and maintenance manual for that outlet. In another example, a maintenance or engineering technician may receive a service call for a medical gas outlet located on the 3^{rd} floor ICU in Room 302. By accessing the electronic annual report and moving to the section of the report covering the 3^{rd} floor ICU, the technician can locate the entry for the outlet in Room 302 and can then select the hyperlink to the operations and maintenance manual for that outlet and can bring the appropriate tools, parts, or equipment to the room on his/her first trip for the service call. This can prevent unnecessary or wasteful repeat trips to bring the proper tools, parts, or equipment. This can also improve the time to service medical gas equipment and help reduce down time and improve patient safety.

One or more methods described herein (e.g., method 200 or 300) can be implemented by computer program instructions or code, which can be executed on a computer. For example, the code can be implemented by one or more digital processors (e.g., microprocessors or other processing circuitry), and can be stored on a computer program product including a nontransitory computer readable medium (e.g., storage medium), e.g., a magnetic, optical, electromagnetic, or semiconductor storage medium, including semiconductor or solid state memory, magnetic tape, a removable computer diskette, a random access memory (RAM), a read-only memory (ROM), flash memory, a rigid magnetic disk, an optical disk, a solid-state memory drive, etc. The program instructions can also be contained in, and provided as, an electronic signal, for example in the form of software as a service (SaaS) delivered from a server (e.g., a distributed system and/or a cloud computing system). Alternatively, one or more methods can be implemented in hardware (logic gates, etc.), or in a combination of hardware and software. Example hardware can be programmable processors (e.g. Field-Programmable Gate Array (FPGA), Complex Programmable Logic Device), general purpose processors, graphics processors, Application Specific Integrated Circuits (ASICs), and the like. One or more methods can be performed as part of or component of an application running on the system, or as an application or software running in conjunction with other applications and operating system.

One or more methods described herein can be run in a standalone program that can be run on any type of computing device, a program run on a web browser, a mobile application (“app”) run on a mobile computing device (e.g., cell phone, smart phone, tablet computer, wearable device (wristwatch, armband, jewelry, headwear, goggles, glasses, etc.), laptop computer, etc.). In one example, a client/server architecture can be used, e.g., a mobile computing device (as a client device) sends user input data to a server device and receives from the server the final output data for output (e.g., for display). In another example, all computations can be performed within the mobile app (and/or other apps) on the mobile computing device. In another example, computations can be split between the mobile computing device and one or more server devices.

Although the description has been described with respect to particular implementations thereof, these particular implementations are merely illustrative, and not restrictive. Concepts illustrated in the examples may be applied to other examples and implementations.

Note that the functional blocks, operations, features, methods, devices, and systems described in the present disclosure may be integrated or divided into different combinations of systems, devices, and functional blocks as would be known to those skilled in the art. Any suitable programming language and programming techniques may be used to implement the routines of particular implementations. Different programming techniques may be employed, e.g., procedural or object-oriented. The routines may execute on a single processing device or multiple processors. Although the steps, operations, or computations may be presented in a specific order, the order may be changed in different particular implementations. In some implementations, multiple steps or operations shown as sequential in this specification may be performed at the same time.

It will be appreciated that the modules, processes, systems, and sections described above can be implemented in hardware, hardware programmed by software, software instructions stored on a nontransitory computer readable medium or a combination of the above. A system as described above, for example, can include a processor configured to execute a sequence of programmed instructions stored on a nontransitory computer readable medium. For example, the processor can include, but not be limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C, C++, C #.net, assembly or the like. The instructions can also comprise code and data objects provided in accordance with, for example, the Visual Basic™ language, or another structured or object-oriented programming language. The sequence of programmed instructions, or programmable logic device configuration software, and data associated therewith can be stored in a nontransitory computer-readable medium such as a computer memory or storage device which may be any suitable memory apparatus, such as, but not limited to ROM, PROM, EEPROM, RAM, flash memory, disk drive and the like.

Furthermore, the modules, processes systems, and sections can be implemented as a single processor or as a distributed processor. Further, it should be appreciated that the steps mentioned above may be performed on a single or distributed processor (single and/or multi-core, or cloud computing system). Also, the processes, system components, modules, and sub-modules described in the various figures of and for embodiments above may be distributed across multiple computers or systems or may be co-located in a single processor or system. Example structural embodiment alternatives suitable for implementing the modules, sections, systems, means, or processes described herein are provided below.

The modules, processors or systems described above can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and/or a software module or object stored on a computer-readable medium or signal, for example.

Embodiments of the method and system (or their sub-components or modules), may be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a PLD, PLA, FPGA, PAL, or the like. In general, any processor capable of implementing the functions or steps described herein can be used to implement embodiments of the method, system, or a computer program product (software program stored on a nontransitory computer readable medium).

Furthermore, embodiments of the disclosed method, system, and computer program product (or software instructions stored on a nontransitory computer readable medium) may be readily implemented, fully or partially, in software using, for example, object or object-oriented software development environments that provide portable source code that can be used on a variety of computer platforms. Alternatively, embodiments of the disclosed method, system, and computer program product can be implemented partially or fully in hardware using, for example, standard logic circuits or a VLSI design. Other hardware or software can be used to implement embodiments depending on the speed and/or efficiency requirements of the systems, the particular function, and/or particular software or hardware system, microprocessor, or microcomputer being utilized. Embodiments of the method, system, and computer program product can be implemented in hardware and/or software using any known or later developed systems or structures, devices and/or software by those of ordinary skill in the applicable art from the function description provided herein and with a general basic knowledge of the software engineering, image processing and/or machine vision arts.

Moreover, embodiments of the disclosed method, system, and computer readable media (or computer program product) can be implemented in software executed on a programmed general purpose computer, a special purpose computer, a microprocessor, a network server or switch, or the like.

It is, therefore, apparent that there is provided, in accordance with the various embodiments disclosed herein, methods, systems and computer readable media for medical gas inspections.

While the disclosed subject matter has been described in conjunction with a number of embodiments, it is evident that many alternatives, modifications and variations would be, or are, apparent to those of ordinary skill in the applicable arts. Accordingly, Applicants intend to embrace all such alternatives, modifications, equivalents and variations that are within the spirit and scope of the disclosed subject matter.

What is claimed is:

1. A tool for conducting medical gas inspections, the tool comprising:
an inspection-item presenting process operating on a computing device, the inspection-item presentation process causing the display of an inspection form corresponding to a selected medical gas asset from among a plurality of medical gas assets being inspected as a facility, the selection of a given asset bringing up a list of selectable inspection items related to the asset, each inspection item being associated with an identifier, wherein each asset is associated with a zone of the medical gas system within the facility;
an information-gathering process also initiated upon the selection of a particular inspection item having a particular identifier, the information-gathering process opening up an inspection form for the particular inspection item selected on the computing device, the form posing standardized questions relating to at least one condition relating to one or more medical gas performance measurements of the given asset, the standardized questions made to be unalterable by a user of the computing device;
an operational pressure test rig interfacing process enabling the computing device to receive medical gas asset performance readings into the inspection form; and
an operations and maintenance manual linking process to link assets in an electronic inspection report with a corresponding operation and maintenance manual such that the link in the electronic inspection report is a hyperlink that permits a user to select the link and automatically access the corresponding operation and maintenance manual section for a corresponding asset.
